(19) United States
(12) Patent Application Publication
(10) Pub. No.: US 2016/0331799 A1
(43) Pub. Date: Nov. 17, 2016

(54) SG 20 minute burn magic formula

(71) Applicant: Steve Gonzales, Long Beach, CA (US)

(72) Inventor: Steve Gonzales, Long Beach, CA (US)

(21) Appl. No.: 15/091,671

(22) Filed: Apr. 6, 2016

Related U.S. Application Data
(60) Provisional application No. 62/179,635, filed on May 15, 2015.

(51) Int. Cl. A61K 36/886; A61K 33/14; A61K 31/19; A61K 9/00; A61K 9/16

(52) U.S. Cl. CPC A61K 36/886; A61K 9/0014; A61K 9/1611; A61K 31/19; A61K 33/14

(57) ABSTRACT

This is a formula in powder form, other burn remedies are a cream. Its use is for minor burns. Once the powder is applied to the wet skin it will draw the liquid from the burned skin preventing a blister from forming, speeds up the healing process and removes all pain in 20 minutes. The ingredients are common household items. Water, vinegar, aloe vera, sodium chloride, baking soda and food coloring. For use at home at work or at play.

There is no federally sponsored or research of development associated with the application. There is no joint research agreement associated with the application.

### FIELD OF THE INVENTION

### BACKGROUND OF THE INVENTION

### BRIEF DESCRIPTION OF SEVERAL VIEWS OF THE DRAWINGS

There are no drawings in this provisional application.

### DETAILED DESCRIPTION OF THE PREFERRED EMBODIMENT

There are no drawings in this provisional application.

### DETAILED DESCRIPTION OF THE INVENTION

The SG 20 Minute Burn Magic Formula is a proven formula to relieve burns to the skin. The formula is described as follows:

SG 20 Minute Burn Magic Yield: Makes 44/ea, 4 oz jars

### Recipe

½ Cup H2O

2 Tbs white vinegar Helps stop sting of the burn

10 grams Aloe Vera Helps skin feel soft and offsets the harshness of sodium and soda

15 drops of food coloring Soothing color—soothing the brain

10 lbs Sodium Chloride Drawing agent—draws viscous through the skin & prevents blistering

1 lb 2 oz baking soda Binder and also helps draw fluid through the skin

### Directions

Split Aloe Vera leaf and scrape out contents. Combine ½ cup water, leaf scrapings, vinegar and food coloring. Bring to a boil, then let simmer 3 minutes. Let the mixture cool to room temperature and then Strain the Aloe out of the cooled liquid mixture before adding it to the sodium. Stir in the 10 pounds of Sodium Chloride and keep mixing until product becomes an even colored green. Spread the mixture out Thinly on trays and let dry in 90 degree enclosure approximately 12 hours. When completely dry, sift in 1 lb 2 oz baking soda while constantly stirring. Sift mixture repeatedly until it becomes uniform in color.

Yield=44/ea 4 oz jars

### Use

Wet afflicted area with tap water and apply Burn Magic until it clings or cakes to the burn area. Wait 20 minutes and rinse. Burn may continue to be painful up to five minutes after application. This is normal and will subside quickly.

The application of this formula works very effectively prior to a blister forming on the skin. Once the powder is applied to the wet skin it will draw the liquid from the burned skin preventing a blister from forming and speeding up the healing process while relieving the pain all within 20 minutes.

1. I claim the SG 20 Minute Burn Magic Formula and process as described.
